(19) United States
(12) Patent Application Publication
(10) Pub. No.: US 2020/0397930 A1
(43) Pub. Date: Dec. 24, 2020

(54) INFECTION RESISTANT BANDAGE SYSTEM

(71) Applicant: Cecil Fred Motley, Rolling Hills, CA (US)

(72) Inventor: Cecil Fred Motley, Rolling Hills, CA (US)

(21) Appl. No.: 16/446,547

(22) Filed: Jun. 19, 2019

(51) Int. Cl. A61L 2/00; A61L 15/16; G06N 3/08

(52) U.S. Cl. CPC A61L 2/0047; A61L 15/16; A61N 5/0624; A61L 2300/404; G06N 3/084

(57) ABSTRACT

This invention, the Infection Resistant Bandage System, is an apparatus and method that uses Ultraviolet C (UVC) and B (UVB) band light to prevent the formation of bacterial biofilms that complicate wound management. The device is a bandage that irradiates the wound site with light at wavelengths of 120 to 270 nm to detect, prevent, and inactivate microorganisms. The device includes a control unit that manages the UV light irradiation protocol, the wavelength selection, the irradiation “on-time”, and the identification of the target bacteria. A Deep Learning Neural Network directs the irradiation protocol and the fluorescence based bacteria detection process. The device is useable in a home, clinical, or emergency field environment. The device efficacy of preventing and eradicating bacterial biofilms is 99%. There are no chemical or antibacterial substances associated with the process.

### PUBLICATION CLASSIFICATION

| | |
|---|---|
| A61K 9/00 | (2006.01) |
| A61P 35/00 | (2006.01) |
| A61K 35/12 | (2006.01) |
| A61P 25/00 | (2006.01) |
| A61K 48/00 | (2006.01) |
| A61N 5/06 | (2006.01) |

### REFERENCES CITED

| | | |
|---|---|---|
| 9,144,690 B2 | Sep. 29, 2015 | McDaniel |
| 6,730,113 B2 | May 4, 2004 | Eckhardt et al. |
| 6,080,189 B2 | Apr. 6, 1998 | |
| 2009/0130169 A1 | May 21, 2009 | Bernstein |
| 2015/0208961 A1 | Jul. 30, 2015 | Duesterhoft et al. |
| 2015/0238774 A1 | Aug. 27, 2015 | Anderson et al. |
| 2013/0064772 A1 | Mar. 14, 2013 | Swiss et al. |
| 2012/0208916 A1 | Aug. 16, 2012 | Cavitt et al. |
| 2004/0034398 A1 | Aug. 15, 2003 | |
| 2011/0200655 A1 | Feb. 16, 2011 | |
| 2013/0274563 A1 | Mar. 12, 2013 | |

### OTHER PUBLICATIONS

⁽¹⁾“The Use of UV Light to Reduce Infections Associated with Central Venous, Arterial, and Urinary Catheters”, Motley et al, CMS Publication, Jul. 16, 2015.
⁽²⁾“UV Inactivation of Pathogenic and Indicator”, Chang et al, APPLIED AND ENVIRONMENTAL MICROBIOLOGY, June 1985, Vol. 49, No. 6, p. 1361-1365.

### BACKGROUND OF THE INVENTION

### 1. Field of Invention

Bacterial biofilms represent a major wound complication associated with surgical procedures, trauma injuries, diabetes, burns, and emergency care and can lead to infections involving several bacteria types both singularly and in groups. Current resistance of bacterial biofilms to drug interventions requires alternative methods and devices for prevention and elimination. In 1985 Chang et al established the ability to inactivate various types of bacteria using ultraviolet (UV) light in the 120 to 270 nm band. Inactivation resulted in preventing the bacteria from reproducing thus causing total elimination.

In 1992 Sen et al. reported that weak electric fields cause biofilms to fail to attach to a surface. A recent study in 2014 has led to a wireless electro-ceutical dressing (WED) based on using silver and zinc printed on a fabric material. This dressing has been FDA cleared and is in clinical use. This technique provides antimicrobial resistance of the surfaces touched by the WED, however surfaces not touch by the bandage are not affected.

Antibiotics and similar drugs, together called antimicrobial agents, have been used for the last 70 years to treat patients who have contacted infectious due to wound complications. Since the 1940s, these drugs have greatly reduced illness and death from infectious diseases. However, these drugs have been so widely used, that various bacteria have become resistant and the antimicrobial agents have become ineffective.

In 2015 Motley et al. reported the effectiveness of using a UV light based device to reduce infections associated with central venous, arterial, and urinary catheters. This device uses ultraviolet C (UVC) and B (UVB) band light to inactivate microbial biofilm on the surface of catheters. The device uses a rotating regiment of wavelengths to irradiate the biofilm which prevents the bacteria from adapting and becoming irradiation resistant.

### 2. Background Art

The management of patients with post-surgical incisions or trauma induced injury is virtually impossible without wound bandages or dressings. Current dressings include gauze, fabric, cotton/tape, antibiotic coated fabrics, and more recently electro-ceutical bandages. Other approaches to preventing and reducing wound infection include open site UV radiation, through bandage light radiation, and electromagnetic radiation.

U.S. Pat. No. 2015/0208961 A1 discloses a method and apparatus that remotely reports information regarding wound dressings. This disclosure does not present a method for detecting and preventing the occurrence of biofilm wound infection.

U.S. Pat. No. 2015/0238774 A1 discloses a method and apparatus for treating psoriasis using UV light. This apparatus only addresses psoriasis and does not provide prevention and elimination of microbial contamination.

U.S. Pat. No. 2013/0064772 A1 discloses a method and apparatus for using an automatically released antibiotic triggered by CO₂ emission at the wound site. This method does not address prevention of the biofilm contamination. Additionally, the microorganisms become resistant to the antibiotic medication.

U.S. Pat. No. 9,144,690 B2 discloses a system and method for using a wide spectrum of light to treat burns, wounds and related skin disorders. This method does not address detection and prevention of the biofilm contamination nor the minimization of keratinocyte destruction.

U.S. Pat. No. 2009/0130169 A1 discloses a method and application for converting electromagnetic energy to ultraviolet C (UVC) radiation for the purpose of deactivating microorganisms. This technique involves using phosphor in the conversion process. The invention does not include a system and apparatus that allows clinical application of the process.

U.S. Pat. No. 6,730,113 B2 discloses a bandage sterilization method that uses ultraviolet light emitted from a lamp. The method also includes sterilization of patient's skin. Light-transmissive film is used to indicate UV exposure. This method does not include automatic control of UV exposure intensity, wavelength, or exposure duration which inactivates the microorganisms while minimizing keratinocyte destruction.

U.S. Pat. No. 2011/0200655 A1 discloses a non-antibiotic coating that when applied to the surface of bandages, wound dressings, and band aides, inactivates microorganisms. This is a chemical base procedure that has no automatic detection or irradiation method.

U.S. Pat. No. 2004/0034398 A1 discloses a method for disinfecting a region of a bandage by selecting a UV light intensity that is irradiated through the bandage material. A UV lamp is used which has no automatic means for controlling the intensity which can lead to keratinocyte destruction.

U.S. Pat. No. 6,080,189 B2 discloses an apparatus for treating a wound by irradiating the wound with infrared light and heat. This method does not address microorganism inactivation or prevention.

Although the aforementioned disclosures use various techniques incorporating the use of UV light in bandages and dressings to address wound contamination, they fail to provide an “in vivo” system and apparatus that automatically detects and eliminates biofilm contamination. The need is especially prevalent for patients requiring long term bandages and dressings in a clinical or outpatient environment.

### SUMMARY OF INVENTION

This invention is an Infection Resistant Bandage (IRB) System that uses ultraviolet UVC and UVB light to resist microbial and viral contamination of trauma, surgical, diabetes and burn wounds “in vivo”. The device includes the ability to detect a wide spectrum of bacterial biofilms by fluorescing the microorganisms with UVB light. UVC light irradiation of the wound site both inactivates and resist further contamination by biofilm bacteria. Light emitting diodes (LED) provide the irradiation source and photo diodes (PD) are used to detect the fluoresced bacteria intensity and spectral characteristics. Deep Learning Neural Networks (DLNN) automatically selects the optimum irradiation protocol, wavelength, intensity, and exposure time.

Exposure to high level UVC radiation at the wound site leads to keratinocyte (skin cells) destruction as well as posing safety conditions for healthcare professional's eyes. The DLNN employed in this invention mitigates this situation by providing the minimum irradiation intensity protocol that inactivates the microorganisms while minimizing the destruction of keratinocytes⁽¹⁾. On the average, a 4 mj/cm² UVC irradiation dose results in a 99.9% bacteria inactivation while causing a 6% keratinocyte destruction⁽²⁾. Eye gear is readily available to protect both patient and healthcare professionals from eye damage.

The IRB system includes an Intel Single Chip Computer (SCC) and associated Graphic Processing Unit (GPU) based control unit attached to a SmartPhone that provides connection to a centrally located processing center where the DLNN are trained and executed. The bandage effectiveness is also monitored and reported to the health care personnel from the central processing center.

### BRIEF DESCRIPTION OF THE DRAWINGS

The invention is illustrated by way of example and not by way of limitation in the figures of the accompanying drawings in which like references indicate similar elements. It should be noted that references to “an” or “one” embodiment in this disclosure are not necessarily to the same embodiment and such references mean at least one.

FIG. 1 illustrates the IRB device configuration.

FIG. 2 illustrates the physical layers of the IRB device.

FIG. 3 illustrates the IRB System Architecture.

FIG. 4 illustrates the IRB Control Unit Architecture.

FIG. 5 illustrates the IRB Deep Learning Neural Network Architecture.

FIG. 6 is a graphical illustration of the Neural Net output layer Sign Activation Function.

FIG. 7 is a graphical illustration of the Neural Net hidden layers ReLu Activation Function.

FIG. 8 is a graphical illustration of the Neural Net input layer Identity Activation Function.

FIG. 9 illustrates the Central Processing Center Architecture.

### DETAIL DESCRIPTION AND CLAIMS

The purpose of the IRB device is to sense the presence of bacterial and/or viral biofilm contamination of wound sites resulting from surgery, physical trauma, diabetes, or burns and eliminate the biofilm using AI managed UVB and UVC light irradiation. The IRB device does not employ chemical coatings or antibiotic regiments, thus avoiding adverse or allergic reactions. It is well known that UV light at 120 to 270 nm wavelengths inactivates microbial bacteria. This inactivation process prevents the bacteria from reproducing thus eliminating it from the infected site. This invention embellishes this process by using AI to manage irradiation protocols that control the wavelength, on-off ratio, and intensity of the light.

1. Biofilm Inactivation Process

UVC light in the range of 120 to 270 nm is strongly absorbed by the nucleic acids of an organism. The light induced damage to the DNA and RNA of an organism often results from the dimerization of pyrimidine molecules. In particular, thymine (which is only found in DNA) produces cyclobutane pyrimidine dimers. When these molecules are dimerized, it becomes very difficult for the nucleic acids to replicate and if replication does occur it often produces a defect which prevents the microorganisms from being viable.

Previous studies have shown that 254 nm is near optimum for germicidal effects on microorganisms. In 1878, Arthur Downes and Thomas P. Blunt published a paper describing the sterilization of bacteria exposed to ultraviolet UV light^{[2]} in the 250 nm to 280 nm range. At these wavelengths, UV light is mutagenic to bacteria, viruses and other microorganisms. This process is similar to the effect of UV wavelengths that produce sunburns in humans. Microorganisms have less protection from UV light and cannot survive prolonged exposure to it.

The primary purpose of this invention is to expose the wound site to a UV wavelength that maximizes the inactivation of biofilm bacteria while minimizing keratinocyte destruction. The DLNN in this invention accomplishes learns which wavelengths, exposure rates, and power level protocol best meets the balance of maximizing bacteria inactivation and minimizing keratinocyte destruction. The protocol is constantly rebalanced as the biofilm contamination is reduced or eradicated. The protocol suspends UV irradiation when no microbial bacteria is detected thus minimizing the keratinocyte destruction.

2. Physical Configuration

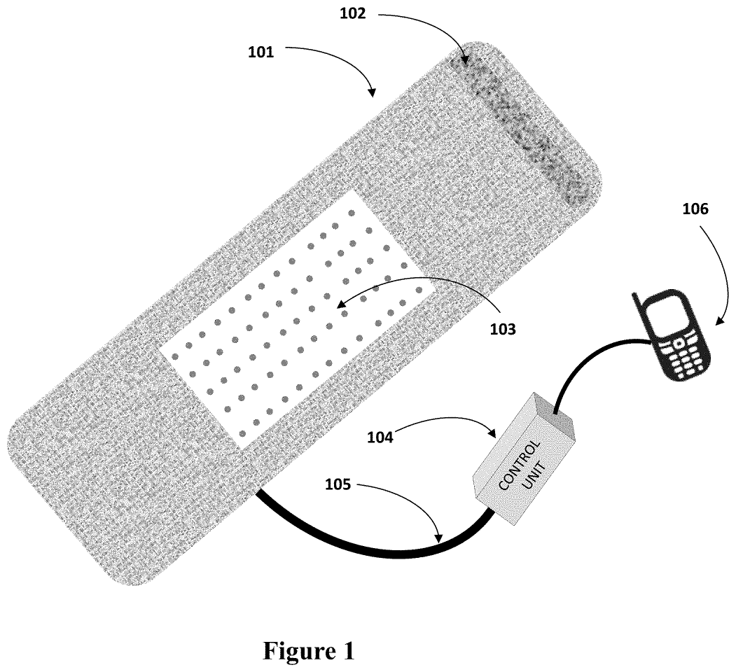

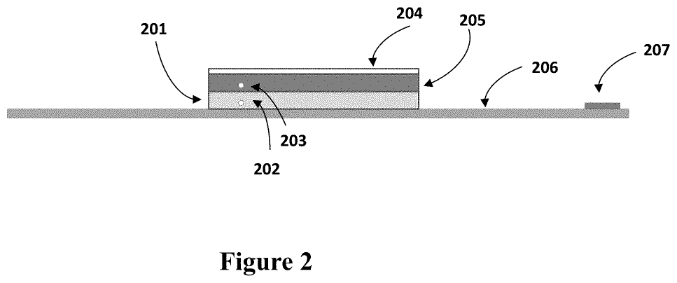

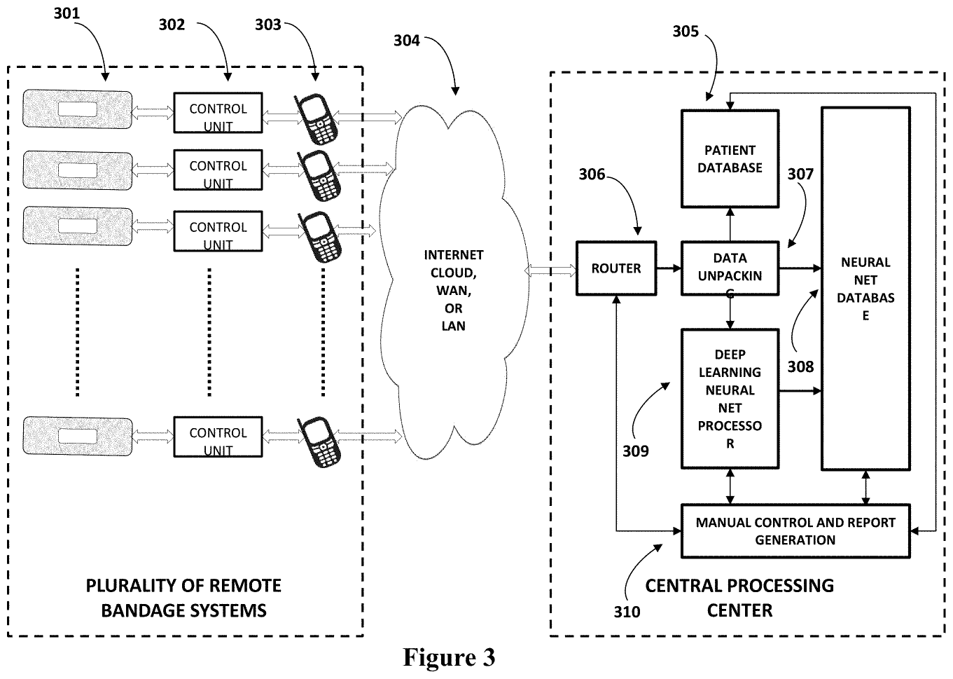

With reference to FIGS. 1 and 2, the physical configuration of the IRB includes a Velcro material wrapping substrate 101, an irradiation surface pad 103, a control unit 104 and a fluid filled optical connection 105. The Velcro material 101 and 206 and associated latch 102 and 207 holds the IRB in place at the wound site and therefore replaces the conventional dressing. The irradiation pad 103 consist of two distilled water filled nutril bladders 201 and 205, and their fluid filled optical couplings 202 and 203. A UV transparent shield material 204 is included for sterile interface to the wound site. One embodiment of this invention includes UV bladder pads from 4 to 12 inches in length and 2 to 6 inches in width and substrate sizes ranging from 4 to 48 inches in length and 2 to 12 inches in width.

3. IRB System Architecture

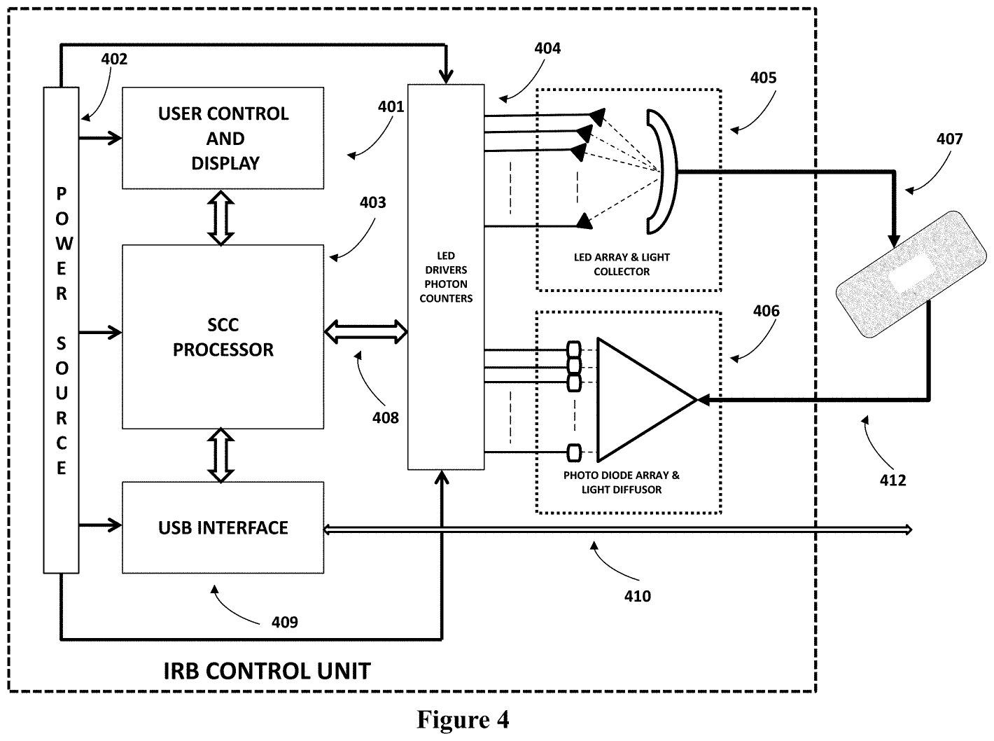

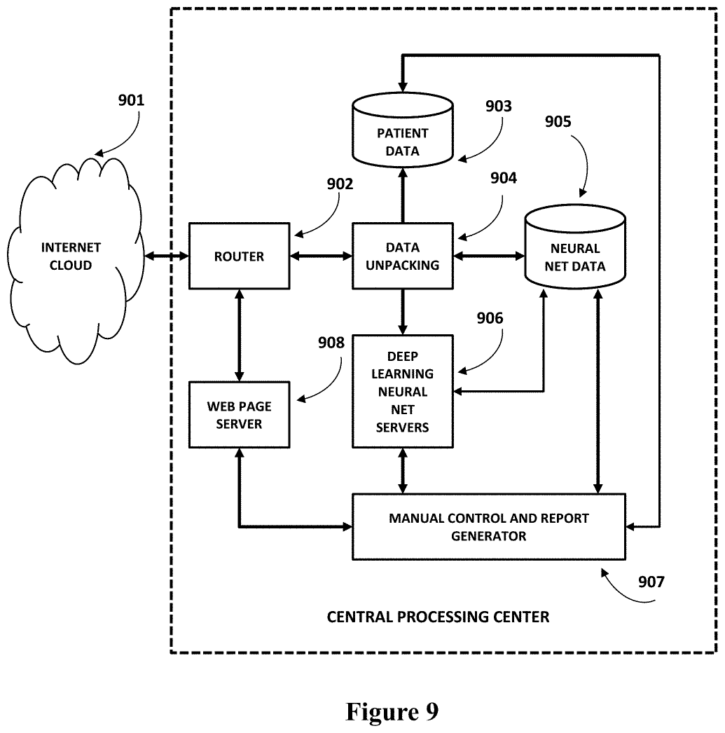

One embodiment of the invention consists of a plurality of IRB remote units connected to a Central Processing Center (CPC) FIG. 9 using the internet cloud FIG. 4. The remote units include the physical bandage 301 and associated fluid filled optical cables, a control unit 302, and a SmartPhone 303. An App on the SmartPhone connects to the CPC where the DLNN algorithms are executed 309. The DLNN uses the fluoresced microorganism biofilm bacteria photon count to determine the irradiation protocol. This protocol is returned to the remote unit's SmartPhone which commands the control unit to execute the directed irradiation protocol. Additionally, the CPC stores patient data 305, the neural database 308, and provides manual control and automatic report generation 310.

A second embodiment of this invention allows standalone operation of an IRB without the need to connect to the CPC FIG. 1. This mode of operation uses the local control unit to execute the neural network DLNN algorithms using the last determined weighting coefficients and biases.

4. IRB Control Unit

The IRB Control Unit architecture is shown in FIG. 4. In one embodiment of the invention the Control Unit provides the interface between the physical bandage 411 and the CPC using a USB connection 409 and 410 to a SmartPhone. In this embodiment, the Control Unit delivers the irradiation 405 energy from eight different LEDs with output wavelengths in the 120 to 270 nm band and receives eight different fluoresced bacteria energy wavelengths from the physical bandage using a photo diode detector array 406 also tuned to eight different wavelengths through fluid filled optical cables 407 and 412. The eight photo diode energy levels are converted to photon counts which are the inputs to the neural network. The local SCC processor 403 manages the operation 401, 408 and 409 in conjunction with the SmartPhone.

In a second embodiment of this invention, the control unit acts as a standalone device for managing the physical IRB. The built in manual control unit 401 allows the health care professional to set the modes and parameters for local standalone operation.

The control unit and its associated fluid filled optical cables are detachable from the IRB irradiation and detector pads and are completely reusable on any size bandage. The control unit is powered by rechargeable batteries or operated directly from an A/C line. In battery mode, the unit is portable and easily worn by the patient.

5. Deep Learning Neural Network

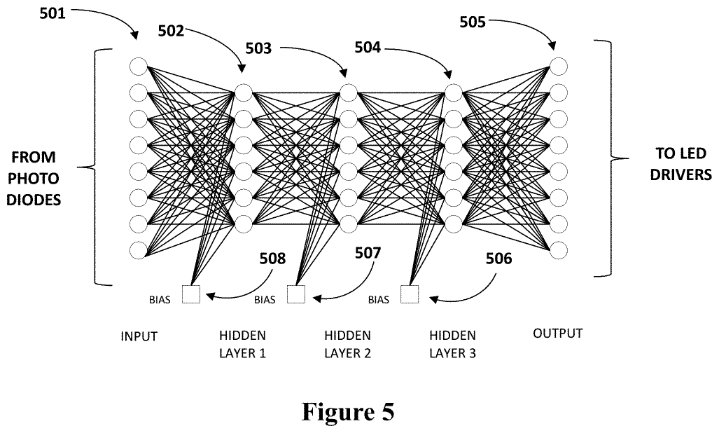

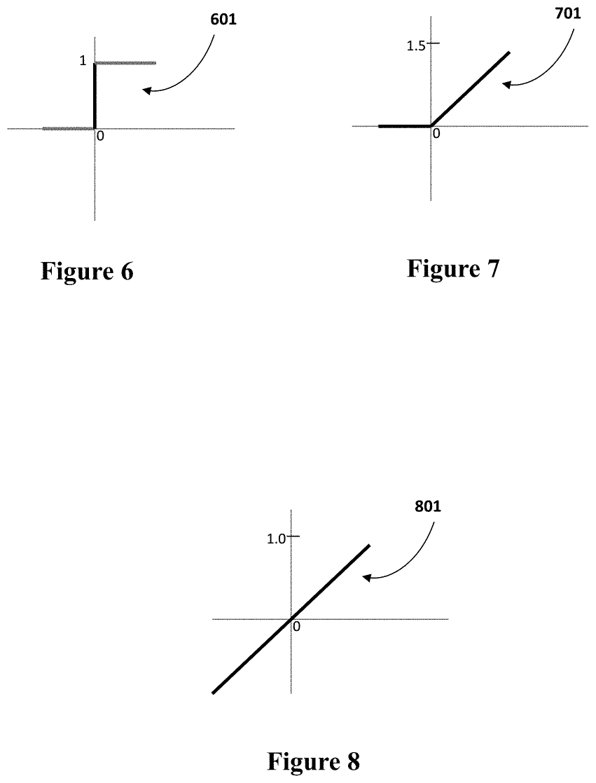

The neural network architecture is illustrated in FIG. 5. In one embodiment of this invention, the DLNN is a feedforward network with eight input 501 and eight 505 output nodes. The input nodes are the photon counts from each photo diode in the detector array 406. The output nodes are the on-off switches for the LED sources 405 used to irradiate the wound site area of the physical IRB. Three hidden layers 502, 503, and 504 with six nodes each are used to learn the weights and biases 506, 507, and 508 that optimize the protocol parameters for each bacteria pattern. The hidden layers use a ReLu Activation Function (FIG. 7) 701 and the output level uses a Sign Activation Function (FIG. 6) 601. The input level uses an Identity Activation Function (FIG. 8) 801. The loss function is least squares regression whose results are used for backpropagation during training.

The IRB units are pre-trained prior to use and are only re-trained when it is necessary to re-calibrate or add new microbial biofilm bacteria pattern information. This process is executed automatically during connection to the CPC using the associated SmartPhone.

A second embodiment of this invention uses the DLNN to determine the on-off time of the UV irradiation. A third embodiment uses the DLNN to determine the intensity of the UV irradiation such that keratinocyte destruction is minimized.

6. Central Processing Center

With reference to FIG. 9, one embodiment of this invention uses a Central Processing Center for a plurality of remote IRB units. The purpose of the CPC is to provide a high speed platform for executing the DLNN and associated patient data 903 management. Supercomputers equipped with Graphic Processing Units (GPU) 906 execute the plurality of neural net instances. The neural net data is stored in a database referenced to patient identification 905. The CPC also provides WEB page hosting 908 services and manual control and report generation 907. All CPC services are accessible from an App on the IRB associated cell phone or PC as well as a workstation at the CPC location.

1. An apparatus comprising:
A wound bandage/dressing that uses ultraviolet light in the UVB and UVC light bands to both detect and eliminate microbial bacteria biofilm “in vivo”. The apparatus detects the type and degree of biofilm contamination from the photon count derived by fluorescing the biofilm bacteria with UVB light. The apparatus irradiates the biofilm with the UVC wavelength light that best inactivates the microbial bacteria. The apparatus cognitively manages this process in real time using Artificial Intelligence Deep Learning Neural Networks. The neural networks determine the type of contamination that is present and applies the UV wavelength, irradiation rate, and intensity required to inactivate the biofilm without destroying keratinocytes. The apparatus components include:
a LED array consisting of eight different wavelengths in the UVB and UVC bands;
a Photo Diode array capable of detecting eight different wavelengths of UV light;
a single chip computer and associated single chip graphics processing unit;
a fluid filled optical transmit cable;
a optical UV light concentrator lens;
a optical UV light collector and diffusor;
a fluid filled optical receive cable;
a Velcro bandage substrate;
a distilled water filled Nutril bladder wound cover pad for UV irradiation;
a distilled water filled Nutril bladder wound cover pad for photon detection;
a Nutril isolation layer;
a Deep Learning Neural Network;
a training data set of wound contamination biofilm patterns;
a validation data set of wound contamination biofilm patterns
a test data set of wound contamination biofilm patterns;
a neural network detection and irradiation algorithm;
a detection protocol algorithm;
a irradiation protocol algorithm and
a rechargeable power source.

2. The apparatus of claim 1 wherein a plurality of Infection Resistant Bandages connect to a Central Processing Center over the internet.

3. The apparatus of claim 1 wherein a plurality of Infection Resistant Bandages connect to a Central Processing Center over a WAN.

4. The apparatus of claim 1 wherein a plurality of Infection Resistant Bandages connect to a Central Processing Center over a LAN.

5. The apparatus of claim 1 wherein the Infection Resistant Bandage operates on a standalone basis.

6. The apparatus of claim 1 wherein a liquid bladder is used to carry UVC light to the Infection Resistant Bandage wound cover pad.

7. The apparatus of claim 1 wherein a liquid bladder is used to carry UVB light to the Infection Resistant Bandages wound cover pad.

8. The apparatus of claim 1 wherein the irradiation “ON TIME”, “POWER LEVEL”, and “WAVELENGTH” are automatically adjusted by the neural network to minimize keratinocyte destruction while insuring up to 99.9% inactivation of the wound biofilm microorganisms.

9. The apparatus of claim 1 wherein the invention is used to bandage wounds caused by surgical procedure.

10. The apparatus of claim 1 wherein the invention is used to bandage wounds caused by physical trauma injuries.

11. The apparatus of claim 1 wherein the invention is used to bandage wounds caused by diabetes.

12. The apparatus of claim 1 wherein the invention is used to bandage wounds caused by burns.

13. The apparatus of claim 1 wherein the irradiation regime (i.e. “WAVELENGTH”, “ON TIME”, “POWER LEVEL”, detection protocol, and pulse rate) is controlled by AI algorithms executed on an embedded processor in the Infection Resistant Bandage control unit.

14. The apparatus of claim 1 wherein the irradiation regime (i.e. “WAVELENGTH”, “ON TIME”, “POWER LEVEL”, detection protocol, and pulse rate) is controlled by AI algorithms executed on a remote central processing center.

15. The apparatus of claim 1 wherein the Deep Learning Neural Network training data, validation data, and test data are collected based on measured biofilm bacteria microorganism patterns.

16. The apparatus of claim 1 wherein the Infection Resistant Bandage substrate sizes range from 4 to 48 inches in length and 2 to 12 inches in width with UV fluid filled bladder sizes of 4 to 12 inches in length and 2 to 6 inches in width.

17. The apparatus of claim 1 wherein a single chip computer and graphic processing unit based control unit is used to execute neural network algorithms that manage the detection and irradiation of bacterial microorganism on the Infection Resistant Bandage cover pad.
